(19) United States
(12) Patent Application Publication
(10) Pub. No.: US 2006/0247670 A1
(43) Pub. Date: Nov. 2, 2006

(54) Lancing device with automatic lancet release

(76) Inventors: Richard W. LeVaughn, Sharpsburg, GA (US); Michael V. Lipoma, Villa Rica, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/415,876

(22) Filed: May 2, 2006

Related U.S. Application Data
(60) Provisional application No. 60/676,896, filed on May 2, 2005.

(51) Int. Cl. A61B 17/32

(52) U.S. Cl. 606/181

(57) ABSTRACT

A lancing device includes a housing, an endcap and a lancet holder within the housing for releasably engaging a lancet. The endcap includes a release mechanism for releasing the lancet from the lancet holder upon separation of the endcap from the housing. In an example embodiment, the endcap is pivotally mounted to the housing, and pivotal opening of the endcap drives a wedge-shaped release member into a slotted sleeve portion of the lancet holder to release the lancet.

### CROSS-REFERENCE TO RELATED APPLICATION

This application claims the priority benefit of U.S. Provisional Patent Application Ser. No. 60/676,896 filed in the United States Patent and Trademark Office on May 2, 2005, which is hereby incorporated herein by reference in its entirety for all purposes.

### TECHNICAL FIELD

The present invention relates generally to lancing devices for medical sampling of body fluids, and more particularly to a lancing device having an automatic indirect removal mechanism for discharging a used lancet from the lancing device.

### BACKGROUND OF THE INVENTION

Various medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics, and in blood typing and screening applications. A lancing device having a spring-energized drive mechanism is often used to carry the lancet along a path of travel, between a retracted or shielded position and an extended position wherein the lancet tip punctures the skin.

Many lancing devices are reusable, and accept a disposable and replaceable single-use lancet. The lancet is typically a relatively small component having a plastic body with a sharp metal needle or blade member embedded therein and having a sharp tip projecting outwardly from the lancet body. The lancet body is typically releasably engaged by a portion of the drive mechanism. For example, a lancet holder portion of the drive mechanism may receive the lancet body with a frictional fit, or may have resiliently opposed members for holding the lancet body in a clamping manner.

Such lancing devices typically require removal of a used lancet for installation of a fresh/sterile one. In removing the lancet, the user must take care not to accidentally stick themself with the sharp tip of the used lancet. Such sticks may only be annoying or painful when the user is the subject whose blood sample was previously taken using the lancet. But they can be life-threatening if the user of the device is accidentally stuck with a lancet tip contaminated with the body fluid of another person who may have a serious disease that is potentially communicable through body fluid transmission.

In addition, many users of lancing devices suffer from impaired visual acuity and/or reduced manual dexterity, rendering manipulation of small objects, such as is often required for the removal of lancets, difficult or inconvenient. This may lead to reduced compliance with a prescribed test regimen, potentially causing adverse health effects.

Thus it can be seen that it would be desirable to minimize or eliminate the requirement for a user to manually manipulate a lancet to remove the lancet from the lancet holder of a lancing device.

Previous attempts to facilitate easier removal of a lancet from a lancing device have included pushing the lancet through the lancet holder, utilizing a custom lancet that has retraction features added, or providing an expanding lancet holder actuated by squeezing “clothes-pin” style features on the lancet holder to release the lancet. Pushing the lancet through the holder can be difficult because it typically requires a feature to be in the way, or take up space inline with the lancet holder when needed, but be out of the way or carried along with the holder for firing. This usually requires extra parts as well, adding to manufacturing expense. Using a custom lancet with features that interact with a release mechanism typically requires a custom/proprietary device that can only use a particular style of lancet. This does not permit the user to take advantage of the ready availability and low cost of standard lancets. And expanding the lancet holder to release the lancet by way of squeezing a “clothes-pin” style clamping holder can often be difficult because of space limitations not allowing adequate room for the user's fingers to operate the squeeze tabs.

Thus it can be seen that needs exist for improvements to lancing devices to provide a mechanism that allows for easier or automatic removal of a lancet from a lancing device. Needs also exist for a method and apparatus allowing indirect removal of a lancet from a lancing device, wherein the user does not need to directly contact the lancet during removal. It is to the provision of an improved lancing device meeting these and other needs that the present invention is primarily directed.

### SUMMARY OF THE INVENTION

In example forms, the present invention relates to a lancing device that enables lancet removal by way of an automatic, indirect release mechanism. The lancing device of the present invention preferably allows the user to release and remove a lancet without touching it. In an example embodiment, the lancing device of the present invention includes a rotating cap having a pivotal finger that acts as a cam or wedge that translates the rotating motion of the cap as it is opened into a spreading or releasing of the lancet holder. This opening of the holder releases the normal force that is required of the holder to keep the lancet in place by friction. During normal operation of the device, when the cap is closed, the wedge is swung back out of the way to allow the lancet holder to securely grip the lancet and move freely through the lancing stroke.

In one aspect, the invention is a lancing device including a housing, an endcap pivotally mounted to the housing, and a lancet holder for releasably holding a lancet, wherein opening the endcap relative to the housing releases the lancet from the lancet holder.

In another aspect, the invention is a lancing device including a housing, an endcap, and a lancet holder within the housing for releasably engaging a lancet, wherein the endcap includes a release member for releasing the lancet from the lancet holder upon separation of the endcap from the housing.

In still another aspect, the invention is lancing device including a housing having an open end, a lancet carrier translationally mounted within the housing and including a split sleeve having a slit therein, and an endcap pivotally attached to the housing by a hinge. The endcap is preferably movable between a closed position covering the open end of the housing and an open position uncovering the open end of the housing. The endcap preferably has a finger that is driven into the slit of the split sleeve of the lancet carrier upon pivoting the endcap into its open position.

And in another aspect, the invention is a method of automatically releasing a lancet from a lancing device, the method including opening an endcap portion of the lancing device to drive an associated release mechanism for releasing, and optionally discharging, the lancet from the lancing device.

In another aspect, the invention is a method allowing for automatic “no additional steps” removal of a lancet from a lancing device. The act of removing or rotating the cap of the device in itself, removes the lancet. The user does not have to perform another operation after cap removal, such as pushing a button or squeezing tabs.

In example forms, the invention takes the form of a simple and elegant mechanism requiring fewer parts than many previously known lancing devices, and is very intuitive and simple for the user to understand and use. The fact that the lancet release function is preferably achieved with no additional controls on example forms of the device is significant, as a device can become very complicated to the user when an additional control is added for implementing additional functions.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a lancing device according to an example form of the invention, with its cap closed.

FIGS. 2a-2c show different views of the lancing device of FIG. 1, with its cap being opened.

FIG. 3 is an exploded view of the lancing device of FIG. 1, showing its various internal components.

FIGS. 4a-4d show a sequence of operation of the lancing device of FIG. 1.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, for example by use of the antecedent “approximately” or “about,” it will be understood that the particular value forms another embodiment.

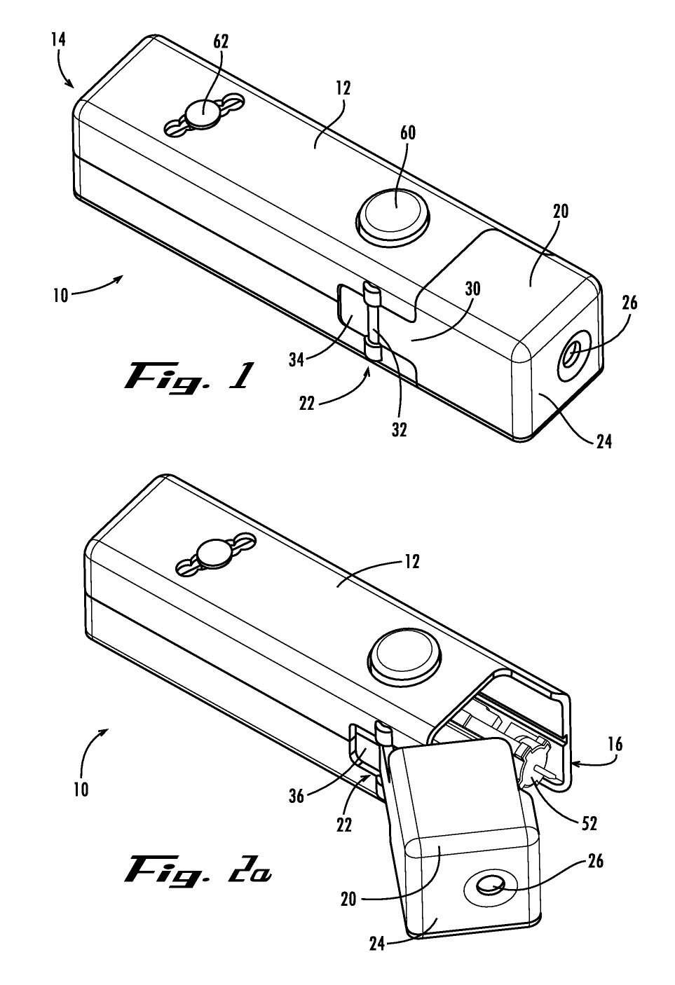

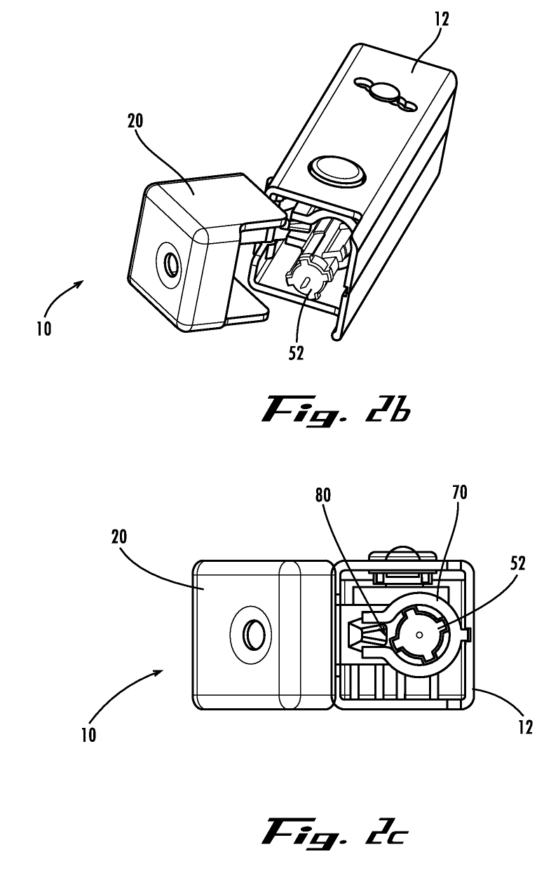

With reference now to the drawing figures, a lancing device 10 according to an example form of the invention is shown. The lancing device 10 preferably comprises an outer housing or shell 12, having a closed distal end 14 and an open proximal end 16. The shell 12 is optionally formed in two halves 12a, 12b, which are joined by snap-fitting, adhesive or solvent bonding, thermal or ultrasonic welding, fastener(s), or other suitable attachment means. An endcap 20 is preferably pivotally mounted by a hinge 22 to selectively cover and uncover the open proximal end of the shell 12. The endcap 20 is preferably pivotal between a closed position (shown in FIG. 1) overlying the open proximal end of the housing, and an open position (shown in FIGS. 2a-2c) exposing the open proximal end of the housing and allowing access to the interior of the housing. The endcap 20 preferably comprises an endface 24 having a lancet passage or opening 26 formed therein, surrounded by a contact face for placement against the subject's skin surrounding the intended lancing site. The opening 26 is preferably aligned along the axis of the lancet tip, and configured to permit the lancet tip to project therethrough upon actuation, to pierce the subject's skin at a desired lancing site. The lancet opening 26 is preferably not so large as to permit the lancet body to pass therethrough, thus preventing launching of a lancet from the lancing device.

The endcap 20 preferably further comprises an arm or flange 30 extending in the direction away from the endface 24. The arm 30 preferably comprises lateral projections forming a hinge pin 32, which are rotationally captured within cooperating bores or recesses 34 in a sidewall of the housing 12, to form the hinge about which the endcap 20 pivots. The distal end of the arm 30 beyond the hinge pin 32 preferably forms a finger 34, which projects pivotally into the interior of the housing 12 as the endcap moves from its closed position to its open position. The housing optionally comprises a cutout section 36, into which the finger 34 fits when the endcap 20 is closed.

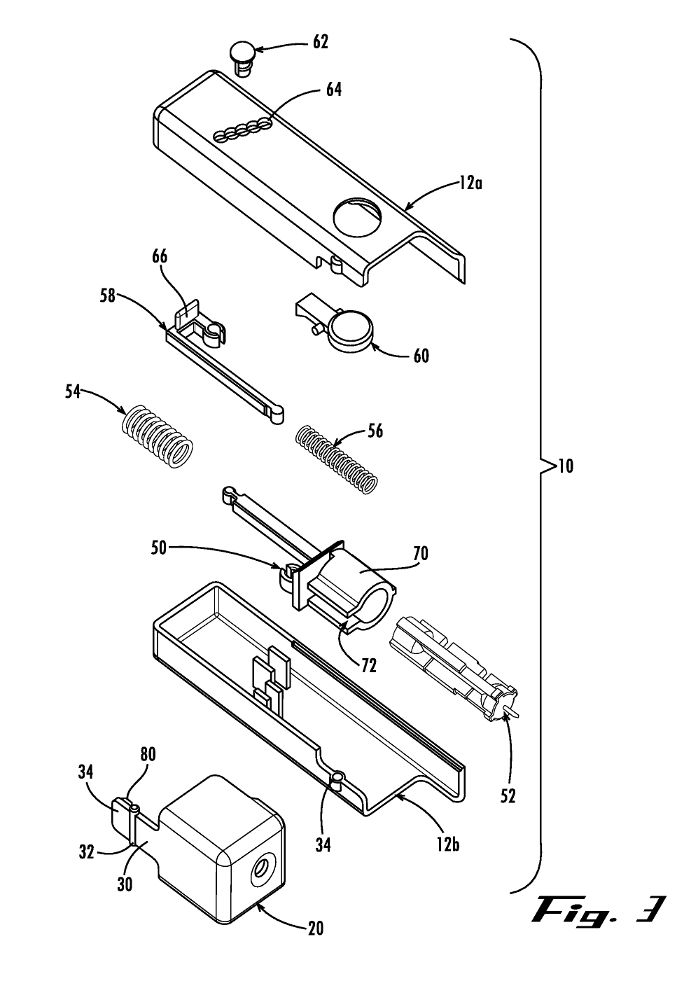

With reference to FIG. 3, the lancing device 10 preferably further comprises a lancet holder or carrier 50 for releasably engaging a disposable lancet 52, most preferably a standard, commercially available lancet. The lancing device 10 preferably also comprises a drive mechanism comprising one or more springs or other biasing means for propelling the lancet along its lancing stroke. In the depicted embodiment, a drive spring 54 propels the lancet from an energized or cocked position within the housing to an extended position with its sharp tip projecting through the lancet opening 26 for pricking the skin at the lancing site, and a return spring 56 returns the lancet to a retracted position with its tip shielded within the housing. In the depicted embodiment, the drive and return springs 54, 56 are coupled to operate in tandem by a retraction linkage 58. The lancet removal mechanism of the present invention, however, is suitable for adaptation and use with various alternate forms of drive mechanisms. An actuation button 60 is provided, for engaging the lancet holder 50 in its energized position until triggered to release the lancet holder to propel the lancet along its lancing stroke under the influence of the drive mechanism. Depth adjustment is optionally provided to allow selective control of the depth of penetration of the lancet tip (i.e., the maximum distance the lancet tip projects beyond the endface 24 of the endcap 20), for example in the form of a depth adjustment tab 62 slidable within an obliquely angled slot 64 in the housing 12, for interaction with a cooperating limit member 66 of the drive mechanism or the lancet. One or more detents formed in the slot 64 optionally provide incremental positioning of the depth adjustment tab 62 in any of a plurality of indexed settings.

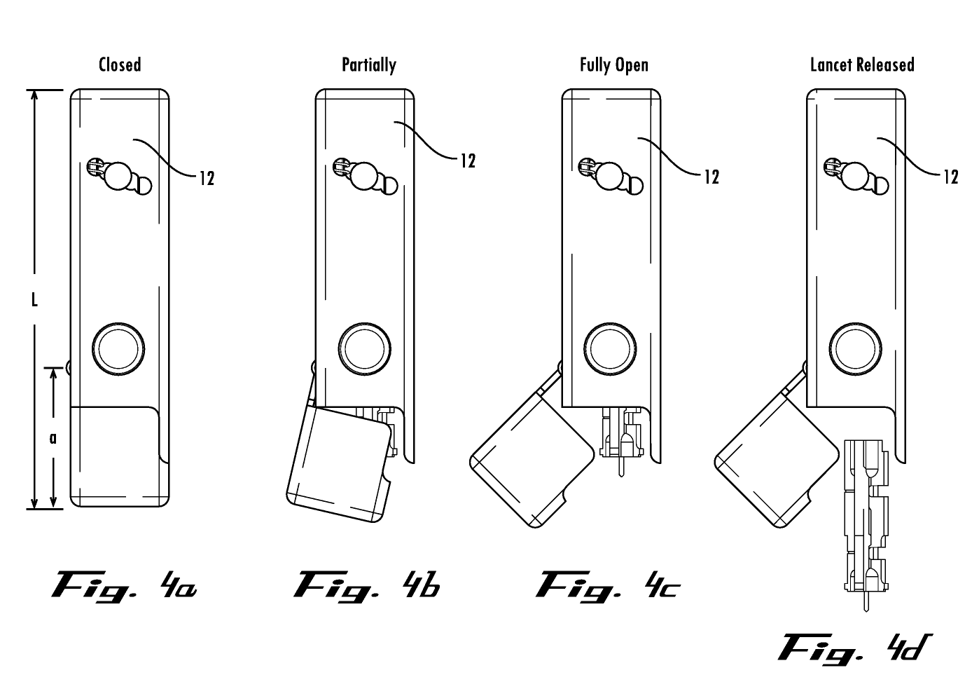

As seen best in FIGS. 2b and 2c, and in the sequential views of FIGS. 4a-4c, opening of the endcap 20 by pivoting it relative to the housing 12 preferably functions as a release mechanism, to automatically release and discharge the lancet 52 from the lancet holder 50, without the need for direct contact with the lancet by the user. For example, in the depicted embodiment, the lancet holder 50 comprises a split-ring sleeve or collar 70 having an open slit or channel 72 along one side. Fins optionally project generally radially outwardly from the sleeve along either edge of the slit 72. The sleeve is preferably formed of a flexible and resilient material, and has an inner dimension configured to securely grip the lancet 52 in its relaxed or released state. Upon pivotally opening the endcap 20, an inwardly-directed wedge portion 80 formed on the finger 34 is driven between the fins, to spread the slit 72 apart, causing the sleeve 70 to flex open and release its frictional grip on the lancet 52. Optionally, a portion of the finger 34 contacts the lancet 52 upon further opening of the endcap 20, to assist in discharging the lancet from the lancet holder. Alternatively, the lancet is discharged under the influence of gravity upon release. The release mechanism optionally comprises a detent mechanism or other means for generating an audible and/or tactile cue just before it releases the lancet, to communicate to the user that the device is in release mode.

In example embodiments, it is preferred that the endcap length (the distance between the endface 24 of the endcap 20 and the pivot point at hinge pin 32, designated a in FIG. 4a) is at least about 20%, and more preferably at least 25%, of the overall length L of the lancing device, in order to provide sufficient leverage to release the grip of the lancet holder upon pivotal opening of the endcap with light to moderate finger pressure actuation force applied on the endcap by the user. It is also preferred that the exterior surface of at least the endcap portion of the lancing device have a flat face on the side opposite the hinge 22 for the user to more easily apply actuation force thereon, in order to facilitate convenient opening of the endcap to release the lancet holder's grip on the lancet. Optionally, the overall device comprises flat external gripping and contact faces on both the hinge side and the side opposite the hinge, for example by providing the housing and endcap with an overall rectangular prismatic external geometry having a generally square cross-section as depicted, for improved ergonomics and ease of use. Ease of application of actuation force to release the lancet may be further enhanced by provision of an endcap length a equal to or greater than the overall device thickness as measured between the hinge side and the side opposite the hinge.

In use, the user opens the endcap 20 and loads a new lancet 52 into the lancet holder 50 by pressing the lancet into the sleeve 70. Pressing the lancet distally serves to energize the drive mechanism and engage the trigger mechanism of the actuation button 60. If a sterile endcap is provided on the lancet, it is removed, as by twisting and pulling. The endcap 20 is closed, and the endface 24 is placed against the subject's skin with the lancet opening 26 over the intended lancing site. The actuation button 60 is pressed to release the drive mechanism, propelling the lancet along its lancing stroke to pierce the skin and permit collection of a blood sample. The used lancet 52 is then removed by pivotally opening the endcap 20, driving the wedge 80 into the slit 72 to flex sleeve 70 open and release its hold on the lancet. The lancet is preferably discharged into a sharps disposal container, and the device is ready for reloading with a new lancet.

While the invention has been described with reference to preferred and example embodiments, it will be understood by those skilled in the art that a variety of modifications, additions and deletions are within the scope of the invention, as defined by the following claims.

What is claimed is:

1. A lancing device comprising a housing, an endcap pivotally mounted to the housing, and a lancet holder for releasably holding a lancet, wherein opening the endcap relative to the housing releases the lancet from the lancet holder.

2. The lancing device of claim 1, wherein the lancet holder comprises a split sleeve, and wherein the endcap comprises a finger for expanding the split sleeve upon opening of the endcap.

3. The lancing device of claim 2, wherein the finger comprises a wedge-shaped portion for contacting cooperating fins of the split sleeve and biasing it open to release the lancet as the endcap is pivotally opened.

4. The lancing device of claim 1, wherein the device has an overall length, and wherein the endcap has a length of at least 20% of the overall length.

5. The lancing device of claim 1, wherein the endcap is connected to the housing by a hinge, and wherein the endcap has a flat contact face opposite said hinge.

6. A lancing device comprising a housing, an endcap, and a lancet holder within the housing for releasably engaging a lancet, wherein the endcap comprises a release member for releasing the lancet from the lancet holder upon separation of the endcap from the housing.

7. The lancing device of claim 6, wherein the endcap is pivotally mounted to the housing.

8. The lancing device of claim 6, wherein the release member comprises a wedge-shaped member.

9. The lancing device of claim 6, wherein the device has an overall length, and wherein the endcap has a length of at least 20% of the overall length of the device.

10. The lancing device of claim 6, wherein the endcap is connected to the housing by a hinge, and wherein the endcap has a flat contact face opposite said hinge.

11. A lancing device comprising:
a housing having an open end;
a lancet carrier translationally mounted within the housing, said lancet carrier comprising a split sleeve having a slit therein; and
an endcap pivotally attached to said housing by a hinge, and movable between a closed position covering the open end of the housing and an open position uncovering the open end of the housing, said endcap having a finger that is driven into the slit of the split sleeve of the lancet carrier upon pivoting the endcap into its open position.

12. The lancing device of claim 11, wherein the finger comprises a wedge for expanding the split sleeve as the endcap is opened to release a lancet from engagement with the lancet carrier.

13. The lancing device of claim 11, wherein the lancing device has an overall length, and wherein the endcap has a length of at least 20% of the overall length of the device.

14. The lancing device of claim 11, wherein the lancing device has an overall length, and wherein the endcap has a length of at least 25% of the overall length of the device.

15. The lancing device of claim 11, wherein the endcap has a flat contact face opposite said hinge.

16. The lancing device of claim 11, wherein the housing and the endcap comprise flat external faces on their hinge sides and on their sides opposite the hinge.

17. The lancing device of claim 11, wherein the housing and the endcap define an overall rectangular prismatic external geometry having a generally square cross-section.

18. The lancing device of claim 11, having an overall device thickness measured between a hinge side of the housing and a side opposite the hinge, and wherein the endcap has a length at least equal to the overall device thickness.

19. The lancing device of claim 11, further comprising a depth-adjustment mechanism.

20. A method of automatically releasing a lancet from a lancing device, said method comprising opening an endcap portion of the lancing device to drive an associated release mechanism for releasing the lancet from the lancing device.
